(19) United States
(12) Patent Application Publication
(10) Pub. No.: US 2006/0167387 A1
(43) Pub. Date: Jul. 27, 2006

(54) Physical activity monitor

(76) Inventors: Horst Buchholz, Solothurn (CH); Urs Mader, Magglingen (CH)

Correspondence Address: OSTROLENK FABER GERB & SOFFEN, 1180 AVENUE OF THE AMERICAS, NEW YORK, NY, 100368403, US

(21) Appl. No.: 11/341,224

(22) Filed: Jan. 26, 2006

(30) Foreign Application Priority Data
Jan. 27, 2005 (EP) 05405041.4

(51) Int. Cl. A61B 5/103

(52) U.S. Cl. 600/595

(57) ABSTRACT

The invention relates to a physical activity monitor comprising means for entering known body data such as size, gender, and weight of a monitor user, means for measuring at least one physiological parameter that is a function of the user's physical activity and serves as an activity indicator, for example heart rate and/or a movement intensity derived from acceleration, a calibration procedure for determining the user-specific sensitivity of the activity indicators and for storing them as a part of the user's body data, as well as an energy expenditure function that allows to determine the user's energy expenditure on the basis of the mentioned body data and of the values of the activity indicators.

The present invention relates to a physical activity monitor according to the preamble of claim 1.

A physical activity monitor, also briefly called “monitor” hereinafter, is an apparatus that allows detecting and monitoring the physical activity related energy expenditure of a user's body. A neutral energy balance, i.e. the balance between food and therefore energy intake on one hand and metabolic energy expenditure on the other hand is an essential requirement for physical well-being. In modern society, which is characterized by technology and consumption, many people do not get enough exercise and therefore more energy is consumed than expended, particularly with the simultaneous tendency to excessive food intake. The consequence is overweight, which leads to health problems of both physical and often also psychic nature.

If the energy balance is positive, body weight increases, if it is negative, it decreases. This known fact leads to the known advice to overweight people that they should exercise more and eat fewer calories. In practice, however, this chronically proves to be difficult as only enduring discipline brings about the desired success and the motivation required therefor is often lacking. This motivation can be decisively strengthened by means of a monitor that continuously monitors energy expenditure and thus enables the user to self-monitor his or her energy balance.

Previously known monitors of this kind include means for entering body related data such as age, weight, and gender, a measuring device for detecting a physiological property related to movement activity, e.g. heart rate or vertical body acceleration, and a data processing unit that determines energy expenditure based on these data by evaluating an energy expenditure function that is normally developed empirically.

It is an object of the invention to provide a physical activity monitor to allows a more accurate assessment of the energy expenditure than the monitors of the prior art.

This object is attained by a monitor having the characteristic features of claim 1. Further objects and preferred embodiments are set forth in the dependent claims.

The energy expenditure of the human body is equal to the sum of the basal metabolic rate and the activity metabolic rate. The basal metabolic rate is a function of the physical constitution, i.e. of body data such as age, weight, and gender, and is essentially defined. In contrast, the activity related metabolic rate is not only a function of the physical constitution but primarily of physical activity. Thus, for calculating the corresponding energy expenditure, information on physical activity is also required in addition to the mentioned body data.

A monitor according to the invention includes means for entering and storing body data of the user and means for measuring one or a plurality of physiological properties related to the physical activity of the user, hereinafter generally designated as activity indicators. Heart rate or a property derived from the vertical body acceleration of a user, i.e. from the acceleration in the longitudinal direction of the elongated body, have been found to be basically appropriate for determining energy expenditure. Surprisingly it has been found that a property which, in combination with heart rate and/or vertical acceleration, is representative of the physical activity, can be derived from the acceleration of the body in the walking/running direction, i.e. from the horizontally forward- or backward-directed acceleration of the vertically standing, upright body. However, other activity indicators, more particularly properties derived from acceleration values measured in other directions, may be used for calculating energy expenditure instead of or in combination with the mentioned ones.

The values of the activity indicators measured at the same moment will generally be designated as indicator tuples hereinafter. The number of values comprised in a tuple corresponds to the number of activity indicators.

The activity indicators measured during a particular activity are not only a function of that activity (e.g. slow walking) but also of the physical constitution of the person performing the activity. Thus, for example, different individuals may exhibit different heart rates during the same physical activity, but the same applies to other activity indicators too. Consequently, the sensitivity of the activity indicators depends on the user of the monitor, i.e. it is user-specific. The values of the activity indicators during one or a plurality of particular reference activities of a user provide information on this user-specific sensitivity of the activity indicators, and taking into account this information allows a more precise assessment of the user's energy expenditure.

By evaluating an information on the user-specific sensitivity of the activity indicators and taking into account this information in the energy expenditure function, the monitor is calibrated for a particular user. For this purpose, the monitor includes a calibration unit intended for measuring the activity indicators during one or a plurality of reference activities of the user, deriving an indicator reference from these indicator tuples (also designated as reference tuples hereinafter) that is representative of the user-specific sensitivity of the activity indicators, and storing it. The indicator reference is a reference object that includes information on the user-specific sensitivity of the activity indicators and that may be constituted by the reference tuples themselves or by another object derived therefrom. The indicator reference is a part of the body data that complements the user's body data such as age, weight, or gender, that have been entered by other means, preferably by a keyboard.

Programmed in the monitor is an energy expenditure function that is usually developed empirically and whose variable values include both one or a plurality of activity indicators and the body data, and among these particularly the indicator reference.

An advantageous approach for the development of an energy expenditure function is based on the realization that the activity-related metabolic rate is greatly affected by the activity type. Based on that fact, a two-part definition of the function enables a systematic approach to its development: First, different activity types are distinguished and a formula for calculating the energy expenditure is developed for each one of these activity types. This formulary forms a first part of the energy expenditure function. The second part of the function is the definition of a procedure for the classification of activities that allows to relate the activity of the user to one of the activity types by means of the values of the activity indicators and of the indicator reference and to select the calculating formula provided for that activity type.

The classification of the user's activity at a particular moment is preferably accomplished by means of the values of the activity indicators measured at that moment. To this end, continuous ranges of value combinations of the activity indicators, hereinafter designated as indicator ranges, are associated to the contemplated activity types. The limits of these ranges are personalized by means of the indicator reference. If the value combination of an indicator tuple is comprised within an indicator range that has been personalized in this manner, the activity is attributed to the activity type for which that indicator range has been defined.

This approach for attributing activities to activity types produces good results particularly when at least two different activity indicators are available. Preferentially, at least heart rate and a parameter derived from the vertical acceleration of the body are used as activity indicators as these allow an accurate distinction of activity types that are relevant in everyday life and have to be treated differently in the calculation of energy expenditure. For example, it is easily determined by these two activity indicators whether the user of the monitor is walking downhill, horizontally, or uphill.

Surprisingly it has been found that the inclusion of the horizontal acceleration of the user measured in the walking direction as a third activity indicator, as a complement to the just mentioned ones, allows a substantial further improvement of accuracy and certainty in the attribution of activities to particular activity types.

The indicator ranges are preferably defined such that they immediately adjoin and jointly cover the range of possible value combinations of the activity indicators so that each activity of the user can be attributed to a contemplated activity type and thus to a formula for the assessment of energy expenditure. Thus, physical activities that are a priori unknown by their activity indicators, i.e. that are not contemplated, will be attributed to the activity type that is closest with regard to the activity indicator values. In this manner, the overall energy expenditure on account of everyday activities can be assessed very accurately by means of the aforementioned preferred activity indicators (heart rate and vertical acceleration).

However, it is also possible to define narrower indicator ranges such that certain value combinations of the activity indicators cannot be attributed to a particular activity type. For estimating the energy expenditure in these cases, a formula that is applicable universally, i.e. independently from the activity type, may be provided for an approximate calculation of the energy expenditure.

The invention will be explained in more detail hereinafter with reference to the figures and by means of an exemplary embodiment thereof.

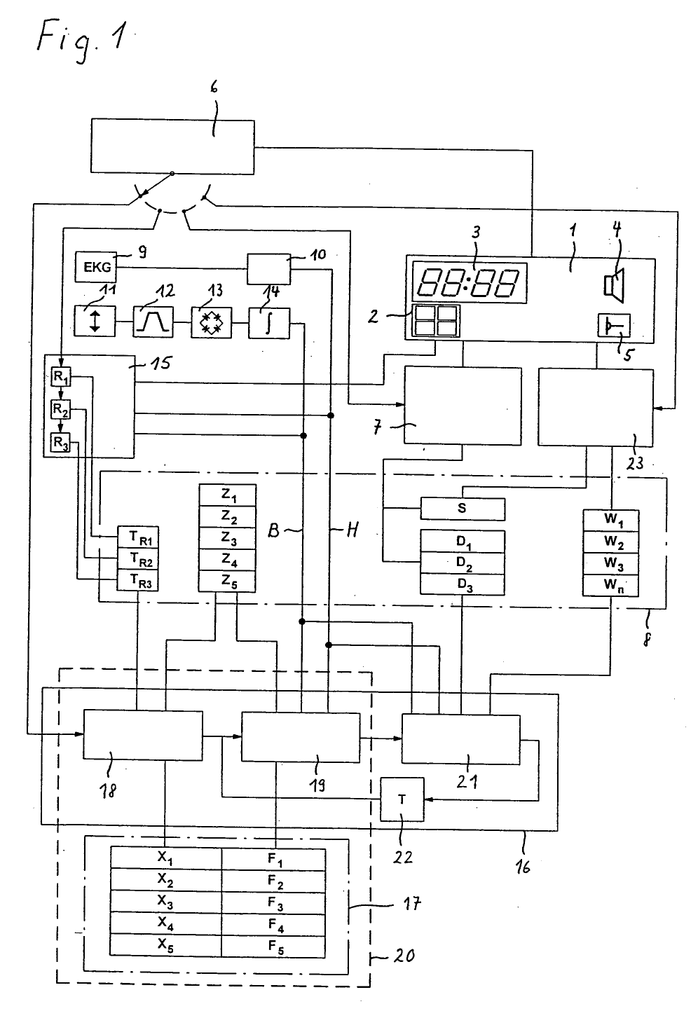

FIG. 1 shows a functional diagram of a monitor of the invention having means for measuring the two activity indicators pulse and acceleration,

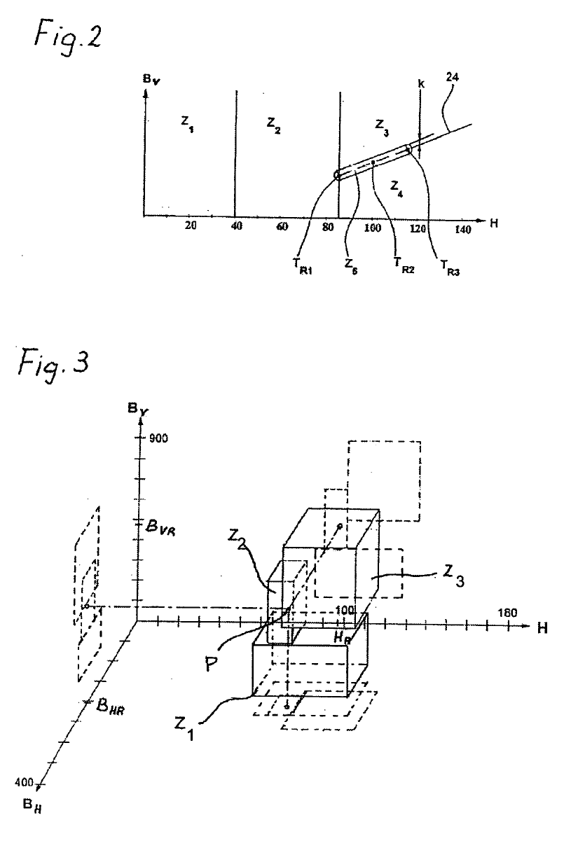

FIG. 2 shows, in a coordinate system spanned by two activity indicators, the points designated therein by the reference tuples and the personalized limits of the indicator ranges,

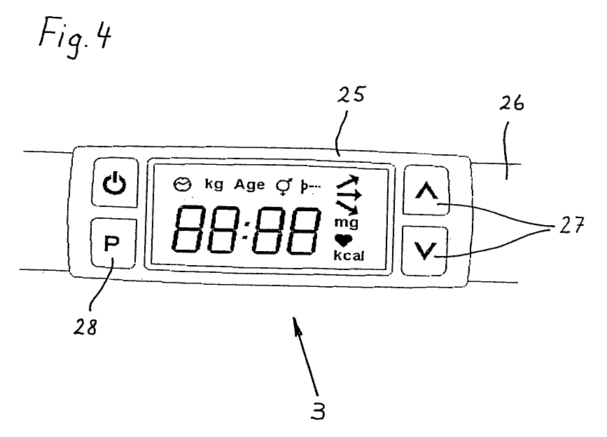

FIG. 3 shows a calibration point and the personalized limits of three indicator ranges in a coordinate system spanned by three activity indicators, and

FIG. 4 shows the enclosure of the monitor attached to a chest strap and its control panel.

The functional diagram shown in FIG. 1 illustrates the functional elements of a physical activity monitor and the way they intercommunicate. Connecting lines without arrows represent signal or information flows, respectively, whereas those with arrows indicate the chronological sequence of different operations. Preferentially, many elements are realized by means of software, which means that the monitor includes a microcontroller that is not represented, however.

The monitor includes an input/output unit 1 with a keyboard 2 for the operation of the monitor, a display 3, an acoustic alarm 4 that is useful particularly in the calibration of the monitor, and a radio or infrared interface 5 for the transmission of data between the monitor and other equipment.

A control unit 6 allows the selection of an operating mode via keyboard 2. The monitor has four operating modes: a programming mode, a calibration mode, a measurement mode and a mode for data analysis.

In the programming mode, a programming unit 7 allows entering data via keyboard 2 that are subsequently stored in a preferably non-volatile data memory 8, for example a battery backed RAM or a so-called Flash Memory. Before using the monitor, general body data D₁, D₂, D₃ of the user, such as his or her age, weight, and gender, which are required particularly for the calculation of energy expenditure, must first be entered and stored. Furthermore, the present example includes the step of programming a value for the projected energy intake S of the user.

A first activity indicator for the assessment of physical activity is heart rate, for whose detection a suitable measuring device is provided. In the present example, the latter consists of an ECG unit 9 and of a following frequency measuring unit 10. ECG unit 9 is generally meant here to designate a unit for measuring at least one cardiac current that allows generating a signal that includes heart rate. A possible alternative for the ECG unit would be a sensor for the detection of pressure waves in the blood whose periodicity also corresponds to the heartbeat. In medicine, the term pulse is used in this context. For the assessment of energy expenditure, the distinction between pulse and heart rate is irrelevant, so that the two terms are used as synonyms here. Frequency measuring unit 10 is preferably designed (or implemented as a part of the monitor software) in such a manner that the current heart rate is always readable at its output. This may e.g. be a counter whose output always displays the number of heartbeats counted during the last 60 seconds. However, for a faster detection of the heart rate, rather than counting the heartbeats during a given period, the interval between two (or the average interval between at least three consecutive heartbeats) is preferably measured and the heart rate is determined by forming the reciprocal value.

As a second activity indicator, a parameter derived from the vertical acceleration of the body is used, for the measurement of which the monitor is provided with an acceleration sensor 11 that is sensitive in the vertical direction. Advantageously, the signal from this sensor 11 is first supplied to a signal filter 12 whose frequency response is such that the acceleration components that are characteristic for physical activities are enhanced and acceleration components that are independent from activity, more particularly body accelerations caused by external influences, are rejected. For signal filter 12, a band-pass filter may e.g. be used. The lower cutoff frequency of such a filter is preferably selected between 0.5 and 1 Hz and the upper cutoff frequency between 5 and 10 Hz.

To provide an activity indicator, the acceleration signal conditioned in this manner is subsequently sent through a rectifier 13, and the rectified acceleration signal is integrated over a time interval by an integrator 14. The length of this time interval is chosen such that the result of the integration is a value that is representative of the type of the movement activity. In the case of a slow, periodical physical activity (e.g. slow walking), the period of the acceleration signal may be greater than 1 second, and the time interval used for the integration should be a multiple of that period. Preferably, the rectified acceleration signal is integrated over a duration of at least 5 seconds. On the other hand, the integrating interval should be as short as possible so that the activity indicator is representative of the type of the physical activity at a certain moment and changes of the activity type in the course of an integrating interval remain an exception. It is therefore advantageous if the integrating interval does not exceed 15 seconds. Preferably, the rectified acceleration signal is integrated by integrator 14 over a duration of 8 to 12 seconds.

For a continuous detection of the physical activity, this evaluation of the activity indicator that is a function of vertical acceleration by means of integrator 14 is periodically repeated, which will be explained in more detail below. Instead of or in addition to acceleration sensor 11 that is sensitive in the vertical direction, an acceleration sensor that is sensitive in the walking/running direction, i.e. to horizontal forward/backward movements, might be used to determine an activity indicator that is a function of the acceleration in the walking/running direction in the same manner.

The passage from analog measurement to digital signal processing may occur at different stages. Preferentially, as many operations as possible are realized by a programmable microcontroller since this allows to perform adaptations in a simple manner by means of software and to reduce the complexity of the analog circuitry. Thus, signal filter 12 is preferably implemented as a digital filter (e.g. an IIR filter). The simplest way to realize integrator 14 is also by means of software: It samples the filtered and rectified acceleration signal periodically and continuously sums up the samples during the integrating interval. The sampling rate of integrator 14 must be significantly greater than the highest frequency component of the signal that is to be integrated and is preferably equal to at least twice the upper cutoff frequency of band pass filter 12 (i.e. a sampling rate of at least 10 Hz in the case of a band pass filter 12 having an upper cutoff frequency of 5 Hz).

Before its first application, in addition to the already described programming operation, the monitor has to be calibrated to the user. In this calibration process, information regarding the user-specific sensitivity of the activity indicators, herein referred to as “indicator reference”, is determined and stored. The information relates to the physical constitution of the user of the monitor and thus represents a part of the body data. If programming the monitor generally designates the input of body data of the user that are required for calculating energy expenditure, then the calibration of the monitor is a part of the programming operation. However, in contrast to the body data (age, weight, etc.) that are programmed via keyboard 2 and are known to the user, the indicator reference has to be derived from the activity indicators that are measured during particular reference activities of the user.

As reference activities, one or several degrees of intensity of locomotion on foot are preferably used. In everyday life, locomotion on foot is the most frequent physical activity, and the resulting personal reference values of the activity indicators are therefore particularly valuable for an accurate calculation of the energy expenditure in the long term (which is therefore primarily dependent on everyday activity). In everyday life, walking is by far the most frequent form of locomotion on foot while other forms such as e.g. jogging or running are limited to mostly shorter phases of sports activity. Consequently, walking is preferably chosen as the reference activity, or different degrees of walking intensity if several reference activities are contemplated.

In calibration mode, the user of the monitor engages in respective reference activities R₁, R₂, R₃ during three calibration cycles. The sequence of these calibration cycles is controlled by a calibration unit 15. The user starts the first calibration cycle by pressing a key on keyboard 2, thereby signalling the beginning of the first reference activity to the monitor. After a period of activity that is long enough to ensure that the activity indicators have reached the values corresponding to the reference activity (which may take several minutes e.g. for heart rate), the current activity indicators appearing at the output of frequency measuring unit 10 resp. of integrator 14 are stored by the calibration unit as a reference tuple T_{R1} that has been determined for the first reference activity R₁. Simultaneously, the calibration unit emits an acoustic signal via acoustic alarm 4 that informs the user of the end of the first reference measurement.

In the same manner, the second and third calibration cycles follow in which reference tuples T_{R2} and T_{R3} determined for reference activities R₂ and R₃ are stored. Particularly when one of the activity indicators is heart rate, the reference activities are preferably performed in the order of their intensities, starting with the reference activity of the lowest intensity (e.g. slow walking). Performing the different reference activities in immediate succession allows minimizing the time during which these reference activities have to be performed by the user. It is therefore advantageous if the mentioned acoustic signal not only informs the user of the end of the first reference measurement but also indicates the beginning of the second reference activity. The same also applies to the following changes in reference activity until all reference measurements are completed. Thus, the user merely signals the monitor the beginning of his or her first reference activity and then passes on to the following more intense reference activity at each acoustic signal. The end of the last reference measurement may be indicated to the user by another acoustic signal.

In this example, the values of the activity indicators determined for the reference activities are directly stored as the indicator reference. Alternatively, however, it would also be possible to derive a separate indicator reference from these reference tuples T_{R1}, T_{R2}, T_{R3} and to store it in the memory, which is explained in more detail below with reference to FIG. 2.

When the calibration is completed, the monitor is operative. In measurement mode, an expenditure assessment unit 16 periodically calculates the energy expenditure W during the last measuring period T and stores the result in memory 8. For this calculation, the expenditure assessment unit 16 may access a memory 17 (which may be a ROM, more particularly the same ROM in which the entire program code of the microcontroller is stored) defining formulas F₁-F₅ for calculating the energy expenditure during five contemplated activity types as well as indicator ranges X₁-X₅ specified for attributing the user's activities to those activity types.

Formulas F₁-F₅ comprise variables including, depending on the activity type for which a formula is intended, one or several ones of the activity indicators and/or one or several parameters from the stored body data such as age, gender, and weight. A possible approach for the development of these formulas is that of regression analysis. To this end, in a group of test subjects, the activity indicators for different degrees of intensity of an activity type (for which a calculation formula is wanted), and in addition, the energy expenditure related to that particular activity is measured, e.g. by means of indirect calorimetry (O₂ and CO₂ measurement). Based on the so determined energy expenditure and on the associated values of the parameters that are taken into account in the formula, the factors by which these parameters contribute to energy expenditure can be determined by multifactorial regression. If a formula takes gender into account, this parameter may be given a value of 1 for a female person and a value of 0 for a male person. In a first approximation, a linear regression model may be used. This procedure is repeated for each one of the activity types distinguished by the monitor, in this example five times, to develop formulas F₁-F₅.

The calculation takes place in three steps, for which expenditure assessment unit 16 is provided with three functional subunits: a personalization unit 18, a classification unit 19, and an evaluation unit 21.

At first, based on the indicator reference, the limits of indicator ranges X₁-X₅ are adapted to the user's physical constitution by means of personalization unit 18. This step will be further explained in detail with reference to FIG. 2. The definitions of the thus personalized indicator ranges Z₁-Z₅ are stored in memory 8. After the calibration of the monitor, the personalization of the indicator ranges is required only once. Therefore, this step might alternatively be performed by the calibration unit following the measurement of the reference tuples, so that a permanent storage of reference tuples T_{R1}, T_{R2}, T_{R3} might be omitted. In this case, the indicator reference would be directly stored as part of the personalized indicator ranges Z₁-Z₅.

Classification unit 19 compares the current activity indicators appearing at the outputs of frequency measuring unit 10 resp. of integrator 14 to the personalized indicator ranges Z₁-Z₅ and thus identifies the indicator range indicated by the indicator tuple. In this manner, the current activity of the user is associated to the activity type for which that particular indicator range is defined. The calculation formula provided for that activity type is then transferred from classification unit 19 to evaluation unit 21. The elements contained in area 20 drawn in dotted lines, i.e. personalization unit 18 and classification unit 19, which are in fact procedures, as well as calculation formulas F₁-F₅ and associated indicator ranges X₁-X₅ jointly form the energy expenditure function of the monitor.

Evaluation unit 21 numerically evaluates the supplied calculation formula and stores the result, a value representing the energy expenditure W that has occurred since the last energy expenditure measurement, in memory 8.

For a continuous assessment of energy expenditure, the last two steps, i.e. the classification of the activity and the evaluation of the formula, are repeated periodically. A timer 22 adjusts the time T between two such measuring cycles to such a short time that physical activities of short duration are also detected. Preferably, the time between two measuring cycles is not longer than 15 seconds. For each one of these cycles, the activity indicator B_{V} dependent on vertical movement activity is determined anew, which means that integrator 14 is also restarted by the same timer 22. The integrator may be a program routine that performs a periodical sampling of the values of the rectified acceleration signal, continuously adds up the samples and outputs the sum after N sampling cycles, thereby initiating an assessment cycle of expenditure assessment unit 16 for calculating the current energy expenditure on the basis of this sum that serves as an activity indicator (and possibly of further activity indicators, in this example heart rate).

For an uninterrupted monitoring of the physical activity, the time T between two measuring cycles is preferably chosen to be equal to the duration of an integrating interval of integrator 14. Integrator 14 continuously samples the rectified acceleration signal and the samples are continuously added up, the sum being output as the new value of activity indicator B_{V} after every N samples, an assessment cycle of expenditure assessment unit 16 being initiated, and integrator 14 being reset. In this case, a separate timer 22 is not necessary since with a defined sampling frequency fₛ of integrator 14, the time T can be determined by suitably selecting the number N of samples included in an integrating interval: T=N/fₛ.

Alternatively, if the acceleration signal is continuously sampled by integrator 14, the latter might be realized in the form of a FIR filter with N filter coefficients, only one of N values from the output data stream of this FIR filter being used as an activity indicator in the variant described above (with T=N/fₛ) . Consequently, in this case, the amount of calculation required for the implementation as a FIR filter would not be justified. However, the application of a FIR filter as an integrator 14 would allow selecting the time T between two measuring cycles shorter than the integrating interval (T<N/fₛ)

Normally, the user is wearing the monitor for a prolonged measuring period, for example from morning to night. After such a measuring period, the user may select the operating mode of the monitor that is intended for data analysis. Via keyboard 2, an analyzing unit 23 allows recalling various information regarding the user's energy expenditure that is subsequently displayed on display 3. Since expenditure assessment unit 16 has stored the energy expenditure values P₁-Pₙ that have been periodically determined during monitoring in memory 8 individually, analyzing unit 23 can not only calculate the cumulated energy expenditure but also analyze and display its progress or transfer the latter to a personal computer via interface 5 for further processing. Further details regarding the evaluation and the display of data are discussed below with reference to FIG. 3.

Ultimately, with respect to FIG. 1, it will be noted that the different functional units such as programming unit 7, calibration unit 15, expenditure assessment unit 16, etc. are preferably elements of a single computer program. For the sake of the simplest possible explanation of the relationships, the different operations in this program are described as functions of distinct units, but the same operations might be grouped and designated differently without modifying their content. Essential are the data processing operations explained with reference to the figure but not their attribution to functional units.

FIG. 2 graphically illustrates the coordinate system spanned by the two activity indicators of the monitor described with reference to FIG. 1, heart rate H being plotted on the horizontal axis and activity indicator B_{V} derived from vertical acceleration on the vertical axis. The indicator tuples designate points in this coordinate system. Marked are the three reference points designated by reference tuples T_{R1}, T_{R2}, T_{R3}. The reference activities R₁, R₂, R₃ for which these reference tuples have been measured are different degrees of walking intensity (slow, faster and fast walking at 2, 4, and 6 km per hour).

It is striking that the indicator tuples corresponding to different degrees of walking intensity designate points lying on a straight line 24 essentially. The position of the point designated by an indicator tuple with respect to that straight line is meaningful with regard to the type of the user's physical activity: If heart rate is below the value measured during slow walking, it may be concluded that the user is motionless. If heart rate is higher, a stronger physical activity is presumable while, based on the assumption that the user's main activity is walking, three cases may at first be distinguished: If the point lies on straight line 24 between reference points T_{R1}, and T_{R3} determined for slow and fast walking, respectively, the user is probably walking. If the point lies below or above straight line 24, the user is presumably walking downhill or uphill, respectively (lower resp. higher heart rate H as compared to straight line 24 for the same movement intensity B).

In this manner, in order to classify the user's activities, continuous ranges Z1-Z5 of the value combinations of heart rate H and movement intensity B_{V} may be defined, the limits of these indicator ranges being at least partially defined by means of the reference tuples.

Straight line 24 is particularly valuable for the definition of the indicator ranges. Therefore, during the calibration of the monitor, an indicator reference is stored that includes the definition of that straight line 24 or, in more general terms, the definition of a calibration curve 24 passing through the points designated by the reference tuples.

The points will rarely lie on a straight line exactly, but the actual relationship between heart rate and movement intensity may be approximated by a straight line so that the amount of calculation required for activity classification remains within reasonable limits. In the case of two reference activities, this straight line is defined by the two reference tuples, and if more than two degrees of walking intensity are contemplated as reference activities, as in the present example, a regression straight line passing through the points of the reference tuples may be used.

A calibration curve is an appropriate reference particularly when it is derived from reference tuples that were measured during reference activities of the same type, in the present example during different degrees of walking intensity. In this case, the calibration curve describes the interdependence of the activity indicators for that activity type. According to the example, it is the function B_{R}(H), which defines the value of movement intensity B_{V} in function of heart rate H for activity type R.

Thus, for the already mentioned activity types, the following indicator ranges Z₁-Z₅ may be defined:

| Activity | Indicator Range |
|---|---|
| Sleeping | X₁: no pulse measured |
| Resting | X₂: 40 Hz < H < H_{R1} |
| Walking downhill | X₃: H_{R1} < H; B_{V} > B_{R}(H) |
| Walking uphill | X₄: H_{R1} < H; B_{V} < B_{R}(H) |
| Walking horizontally | X₅: H_{R1} < H < H_{R3}; B_{V} = B_{R}(H) +/− k |

If no pulse can be measured it must be assumed that the user has taken off the monitor. If the monitor is only taken off for sleeping, a formula provided for calculating energy expenditure while sleeping may be used for indicator range X₁. In this manner it is possible to measure the user's energy expenditure continuously for several days and even weeks, provided that the monitor is only taken off during the sleeping periods.

In this table, the indicator ranges are defined by means of variables for the values of the reference tuples and thus in a general form. They are therefore not designated Z₁-Z₅ as in the figure but X₁-X₅. The indicator ranges might be defined in this form in memory 17 of the monitor mentioned with reference to FIG. 1, for example. The adaptation of these ranges to the user of the monitor, designated as personalization, is accomplished by replacing the variables by actual values that are known from the indicator reference.

As mentioned above, an activity type that is dominant in everyday life is preferred for the reference activities, and for the same reason it is advantageous also to develop a special formula for calculating the energy expenditure during that activity type. In this respect, the indicator range intended for the attribution of activities to the reference activity type is preferably defined by a tolerance value k for the maximum deviation from calibration curve 24.

An alternative for the definition of indicator ranges X₁-X₅ by means of abstract rules or formulas according to the above table consists in specifying concrete, i.e. numerically determined standard indicator ranges X₁-X₅ with reference to a standard calibration curve of an average user. Then, by a transformation of the range limits, personalized indicator ranges Z₁-Z₅ are derived from the latter whose position with respect to the calibration curve of the user corresponds to the position of the corresponding standard indicator ranges with respect to the standard calibration curve.

Ultimately, it will be mentioned with reference to FIG. 2 that the mentioned activity types might be defined in another manner, thereby resulting in a different, possibly more complex shape of the indicator range limits. Also, in particular, a different definition of the indicator ranges may result if the monitor is to distinguish a greater number of activity types. The described method for activity classification is also applicable analogously if an activity indicator that is a function of the body acceleration in the walking/running direction (or another activity indicator) is used instead of activity indicator Bv that is a function of vertical body acceleration. Alternatively, with a corresponding adaptation of the indicator range definitions, the latter might be used instead of heart rate. All three mentioned parameters (heart rate and acceleration both vertical and in the walking/running direction) may be used as activity indicators, two of them being used for activity classification in the described manner and the third one (and possibly additional ones) only entering into the calculation of energy expenditure as variables contained in formulas F₁-F₅.

In a further improved embodiment of the monitor, in addition to the two activity indicators used in the previously described example, i.e. heart rate and vertical acceleration, the horizontal body acceleration measured in the walking/running direction is also taken into account as a third activity indicator not only in formulas F₁-F₅ for the calculation of energy expenditure but also for the purpose of activity classification. The functional diagram of this monitor corresponds to that shown in FIG. 1 whereas in addition to acceleration sensor 11, a non-represented acceleration sensor for the measurement of horizontal body acceleration is provided that yields an activity indicator derived from horizontal acceleration in the same manner via a signal filter, a rectifier and an integrator.

In the calibration of the monitor, the associated activity indicators for three reference activities R₁, R₂, R₃ are determined as described with reference to FIG. 1. The first reference activity R₁ is slow walking (strolling, slow promenading). The second reference activity R₂ is moderate walking, which corresponds to goal-oriented walking, e.g. from one place to another. The third reference activity R₃ is fast walking, where the user is asked to walk briskly. Each one of these reference activities is performed for about 3 minutes and the values of the activity indicators measured during the last minute of each activity are averaged and stored as reference tuples T_{R1}, T_{R2}, T_{R3}.

Here also, in measurement mode, energy expenditure W is periodically assessed by expenditure assessment unit 16. Memory 17 includes—in contrast to FIG. 1—energy expenditure calculation formulas F₁-F₃ for three defined activity types and indicator ranges X₁-X₃ specified for attributing the user's activities to those activity types. In addition, memory 17 includes a universal energy expenditure calculation formula (not shown in FIG. 1) that is used when the user's activity cannot be attributed to any one of the predetermined activity types on the basis of the measured activity indicators. In analogy to what has been said with regard to FIG. 1 about the formulas relating to particular activity types, this universal formula may also be developed by means of regression analysis, the analysis preferably being based on the entirety of the measuring data used for the development of activity specific formulas F1-F3.

The calculation of energy expenditure is accomplished as described with reference to FIG. 1, the personalization of indicator ranges X₁-X₃ by personalization unit 18 and the following classification of the user's activity by classification unit 19 being explained in more detail below with reference to FIG. 3.

FIG. 3 shows the coordinate system spanned by the three activity indicators heart rate H, vertical acceleration B_{V}, and horizontal acceleration B_{H}, heart rate H being plotted on the horizontal axis, activity indicator B_{V} derived from vertical acceleration on the vertical axis, and activity indicator B_{H} derived from horizontal acceleration on the third axis. The indicator tuples designate points in this coordinate system. A reference point in this coordinate system that will be designated as calibration point P hereinafter is defined by the arithmetic mean H_{R}, B_{VR}, B_{HR} of the values measured for reference activities R₁, R₂, R₃ in the calibration process of the monitor.

The extent of the personalized indicator ranges as measured by the values of the three activity indicators is the same for all users; they are not adapted during the personalization of the indicator ranges X₁-X₃ stored in memory 17 (FIG. 1). The same applies to the location of the indicator ranges in the coordinate system with respect to the calibration point. Each one of indicator ranges X₁-X₃ is defined by value intervals indicated in relation to the calibration point and stored in memory 17 in a tabular form:

| | Indicator Range | | | |
|---|---|---|---|---|
| Activity | | H | B_{V} | B_{H} |
| Slow walking | X₁ | −9 to 31 | −339 to −102 | −52 to 32 |
| Moderate walking | X₂ | −7 to 4 | −119 to 180 | −20 to 22 |
| Fast walking | X₃ | 4 to 41 | 18 to 410 | −36 to 58 |

Heart rate is indicated in Hertz while the values of the other two activity indicators are values that are derived from the respective accelerations and representative of the movement intensities and whose measurement has been explained in detail with reference to FIG. 1. These indicator ranges were empirically determined while care was taken that the ranges do not overlap.

For the personalization of indicator ranges X₁-X₃, the mentioned calibration point is first calculated by personalization unit 18 on the basis of reference tuples T_{R1}, T_{R2}, T_{R3} stored as the indicator reference. The calibration point marked in FIG. 3 is at H_{R}=97 Hz, B_{VR}=488, and B_{HR}=202. The limits of the personalized intervals, which define the personalized indicator ranges Z₁-Z₃, are subsequently determined by displacing the intervals defining indicator ranges X₁-X₃ by these values.

After the personalization of the indicator ranges, classification unit 19 periodically determines the personalized indicator ranges Z₁-Z₃ indicated by the current values of the activity indicators and selects the associated energy expenditure calculation formulas F₁-F₃. An indicator range Z₁-Z₃ is unequivocally indicated when the values of all three activity indicators are comprised within the corresponding value intervals defining the personalized indicator range. If the point in the coordinate system that is defined by an indicator tuple is comprised in none of the (personalized) indicator ranges, the universal formula provided for this case may be employed for the approximate calculation of energy expenditure.

As an alternative thereto, in the case of lack of unambiguity, it may be verified whether an unambiguous association was possible in the preceding detection cycle. If this is the case, the formula intended for this activity type is again selected instead of the universal formula if at least two of the activity indicators are again comprised within the personalized value intervals defining this indicator range. This error correction procedure may also be extended to the second and further detection cycles following a detection cycle in which an unequivocal attribution was possible.

To complement indicator ranges Z₁-Z₃ represented in FIG. 3, additional indicator ranges for further activity types and associated energy expenditure calculation formulas might be provided. In this example, indicator ranges Z1-Z3 are defined by value intervals of the activity indicators, thereby offering the advantage that the indication of such a range by an indicator tuple can be verified by simple comparison operations. However, more complex definitions of one or several indicator ranges are also possible whose personalization may be accomplished analogously by displacing the point of origin with respect to the position of the calibration point.

FIG. 4 shows a possible design of a monitor having the functionality described with reference to FIG. 1. The monitor has an enclosure 25 mounted on a chest strap 26. The latter is worn by the user under his or her clothes directly on the skin. Enclosure 25 contains vertically sensitive acceleration sensor 11, and the non-represented electrodes of ECG unit 9 are arranged on the chest strap. Further sensors for the detection of other activity indicators, more particularly an acceleration sensor that is sensitive in the walking/running direction, may also be contained in enclosure 25. Several keys 2 are provided for operating the functions of this monitor. One of these keys 28 is intended for selecting the operation mode of the monitor, and the two arrow keys 27 allow selecting different functions of the monitor that are displayed on display 3 or adjusting the values of body data such as age or body weight in the programming procedure.

The measurement mode and the mode for data analysis have been described as separate operating modes with reference to FIG. 1. However, data analysis and particularly the continuous display of the cumulated energy expenditure are also possible during a measurement phase so that information regarding the user's current energy expenditure or energy balance status is available at any time.

In addition to the periodically measured energy expenditure values W, analyzing unit 23 (FIG. 1) also needs information regarding energy intake for calculating the energy balance. Theoretically it would be possible to provide means for entering all meals with the respective calory values. However, this procedure is very laborious for the user so that is preferably omitted. It is sufficient to program a nominal value S (FIG. 1) for energy intake, i.e. a value for the total projected energy intake during a day (or another time period). A continuous intake of this quantity of energy during the day is preferably assumed for this purpose. In other words, for calculating the energy balance at the hour x, the preceding energy intake is calculated as S/24*x and this value is compared to the measured, cumulated energy expenditure.

The fiction of a continuous energy intake offers the additional advantage of allowing not only the calculation of the energy balance since the beginning of the measuring period but also the calculation and display of short-term trends of that energy balance. Thus, it is possible at any time to calculate the energy intake during an analysis period lasting for the preceding n hours as S/24*n and to compare the resulting value to the cumulated energy expenditure for that same analysis period. The duration of the analysis period may be programmable by the user, and the short-term energy balance trend obtained as a result is preferably displayed on display 3 by means of trend arrows 28.

1. Physical activity monitor comprising a memory 8 and means for entering first body data of a user into that memory, means for measuring one or a plurality of physiological properties that are dependent on the user's physical activity and serve as activity indicators, a data processing unit for determining the user's energy expenditure (P) on the basis of the body data and of the values of the activity indicators by evaluating an energy expenditure function, and a display for displaying at least one item of information related to that energy expenditure (P), characterized in that a calibration unit for controlling the sequence of a calibration procedure is provided by which the activity indicators are measured during one or a plurality of reference activities (R1, R2, R3) of the user, and an indicator reference indicating the user-specific sensitivity of the activity indicators is derived from these reference values (TR1, TR2, TR3) and stored as a complement to the first body data.

2. Physical activity monitor according to claim 1, wherein that the means for measuring the activity indicators include a sensor for measuring the heart rate (H) and/or a sensor for measuring vertical body acceleration, the measured heart rate and a property (BV) derived from vertical body acceleration serving as activity indicators, respectively.

3. Physical activity monitor according to claim 1, wherein that the reference activities are at least two different degrees of intensity of locomotion on foot, preferably of walking.

4. Physical activity monitor according to claim 1, wherein that the energy expenditure function includes a plurality of calculating formulas (F1-F5) for different activity types and a procedure for activity classification, by which procedure the user's activity is attributed to one of the activity types on the basis of the activity indicators and of the indicator reference and the calculation formula intended for that activity type is selected.

5. Physical activity monitor according to claim 4, wherein that the energy expenditure function includes a universal energy expenditure calculation formula for values of the activity indicators to which the procedure for activity classification attributes no specific activity type.

6. Physical activity monitor according to claim 4, wherein that continuous ranges (X1-X5) of value combinations of the activity indicators are associated to the activity types, and the data processing unit is programmed to personalize the limits of these indicator ranges (X1-X5) on the basis of the indicator reference, to identify a personalized indicator range (Z1-Z5) indicated by the values of the activity indicators, and to determine the current energy expenditure using the calculating formula (F1-F5) associated thereto.

7. Physical activity monitor according to claim 6, wherein that the indicator ranges are defined by continuous ranges of the value combinations of two activity indicators, preferably of heart rate (H) and of the property (BV) derived from vertical body acceleration.

8. Physical activity monitor according to claim 6, wherein that the indicator ranges are defined by continuous ranges of the value combinations of three activity indicators, preferably of heart rate (H), of a property (BV) derived from vertical body acceleration, and of a property (BH) derived from the horizontal body acceleration in the walking/running direction.

9. Physical activity monitor according to claim 6, wherein that the calibration procedure includes the measurement of the activity indicators during at least two and preferably three reference activities and the indicator reference includes the definition of a calibration curve in a coordinate system spanned by at least two activity indicators which essentially runs through the points designated by the reference values (TR1, TR2, TR3) of the activity indicators, the indicator ranges being defined by ranges in this coordinate systems of which at least one is partially defined by the shape of the calibration curve.

10. Physical activity monitor according to claim 9, wherein that an energy expenditure calculation formula (F5) is provided for the activity type of the reference activities (R1, R2, R3) and the definition of the indicator range (X5) intended for attributing activities to that activity type includes a tolerance value (k) for the maximum deviation of the point designated by the activity indicators from the calibration curve.

11. Physical activity monitor according claim 6, wherein that the indicator reference includes a calibration point designated by averages of the reference values (TR1, TR2, TR3) of the activity indicators in a coordinate system spanned by at least two activity indicators, the indicator ranges being defined by ranges in this coordinate systems of which at least one is partially defined by the location of the calibration point.

12. Physical activity monitor according to claim 11, wherein that the calibration point is defined by the arithmetical mean of the reference values (TR1, TR2, TR3) of the activity indicators.

13. Physical activity monitor according to claim 11, wherein that one or several ones of the indicator ranges and preferably all indicator ranges have a fixed size and their positions in the coordinate system are defined with respect to the location of the calibration point.

14. Physical activity monitor according to claim 13, wherein that the positions of the indicator ranges in the coordinate system are defined by predetermined distances from the calibration point.

15. Physical activity monitor according to claim 1, wherein by means for entering the targeted energy expenditure (S) and a display for the display of the short-term and/or the long-term energy balance.

16. Physical activity monitor according to claim 15, wherein that the step of programming a value for the average long-term energy intake rate (S) is included and that for calculating the energy balance for a short and/or a long measuring period (x), the product of the average energy intake rate (S) and of the duration of the measuring period (x) is assumed as the energy intake during that period (x) and compared to the cumulated energy expenditure measured for that same period (x).
